(19) United States
(12) Patent Application Publication
(10) Pub. No.: US 2024/0325627 A1
(43) Pub. Date: Oct. 3, 2024

(54) INTRAVENOUS FLUID LINE CLIP

(71) Applicant: Specian Services, LLC, Jupiter, FL (US)

(72) Inventors: David W. Specian, Jupiter, FL (US); Anthony Dowdy, West Palm Beach, FL (US)

(21) Appl. No.: 18/619,499

(22) Filed: Mar. 28, 2024

Related U.S. Application Data
(60) Provisional application No. 63/492,813, filed on Mar. 29, 2023.

(51) Int. Cl. A61M 5/14

(52) U.S. Cl. CPC A61M 5/1418

(57) ABSTRACT

A surgical clip that secures to an intravenous fluid (IV) infusion main line, thereby preventing separation of two connected fluid lines. The surgical clip comprises a first end, a second opposing end, and a main body separating the first end and the second opposing end by a length. The surgical clip comprises a first fluid line engagement member located at one end of the main body and a second fluid line engagement member located at a second end of the main body. Use of the surgical clip minimizes leaking and prevents unintentional patient awareness during a surgical procedure or inadequate perfusion resulting from provider error (failure to properly secure two connections) or disconnections at port sites.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/492,813, entitled “INTRAVENOUS FLUID LINE CLIP”, filed Mar. 29, 2023. The content of the above referenced application is incorporated herein by reference in its entirety.

### FIELD OF THE INVENTION

Embodiments of the present invention relate to a medical device; to a medical device used primarily in surgical procedures; and more particularly, to a surgical clip that secures to an intravenous fluid infusion line, thereby preventing separation of two connected fluid lines.

### BACKGROUND OF THE INVENTION

Surgical procedures are critical components of health care around the world. The number of surgical procedures performed is expected to rise over the coming years. Surgical procedures often require anesthesia in which anesthetics are administered to patients to induce a sleep-like state during the procedure. Use of anesthesia controls pain, and can help regulate other important physiological measurements, such as breathing or blood pressure. As an integral part of the surgical procedure, the effects of the anesthetics on the patient are continuously monitored to ensure a patient does not “wake up” while the medical procedure is being performed.

Administering medications to a patient, whether during a surgical procedure or post-surgical procedure, is usually accomplished through intravenous fluid/drug infusion lines. Within an intravenous fluid/drug infusion line there are multiple injection sites, thus allowing the surgical team to inject different types of fluids as needed, thus providing a single main line with multiple connecting lines. At each of these injection sites, a connection means is provided to ensure that the fluid that is to be injected is connected, and remains connected, to the main line. Such connection means are not fail-proof. An unconnected line could result in the patient not receiving the proper dosage of the medicine during the surgical procedure. Preventing disconnection of any one of the multiple lines from the main line is critical for patient safety.

### SUMMARY OF THE INVENTION

Embodiments of the present invention relate to a surgical clip that secures to an intravenous fluid infusion main line, thereby preventing separation of additional, independent, fluid connecting lines. The surgical clip comprises a first end, a second opposing end, and a main body separating the first end and the second opposing end by a length. The surgical clip is constructed and arranged to attach and secure to a tube capable of carrying fluid via a first fluid line engagement member located at one end of the main body and a second fluid line engagement member located at a second end of the main body. Use of the surgical clip is designed to minimize leaking and prevent patient awareness or inadequate perfusion resulting from provider error (failure to properly secure two connections) or disconnections at port sites.

Accordingly, it is an objective of the invention to provide a medical device that improves patient safety during a medical procedure in which fluids are infused into the patient prior to a surgical procedure, during the surgical procedure, or after the surgical procedure.

It is a further objective of the invention to provide a medical device which decreases the risk of provider error during a medical procedure in which fluids are infused into the patient prior to a surgical procedure, during the surgical procedure, or after the surgical procedure.

It is yet another objective of the invention to provide a medical device which minimizes the risk of provider error during a medical procedure in which fluids are infused into the patient prior to a surgical procedure, during the surgical procedure, or after the surgical procedure.

It is yet another objective of the invention to provide a medical device which prevents provider error during a medical procedure in which fluids are infused into the patient prior to a surgical procedure, during the surgical procedure, or after the surgical procedure.

It is a still further objective of the invention to provide a medical device which reduces the risk of infusion line disconnection at port sites during a medical procedure in which fluids are infused into the patient prior to a surgical procedure, during the surgical procedure, or after the surgical procedure.

It is a further objective of the invention to provide a medical device which minimizes the risk of infusion line disconnection at port sites during a medical procedure in which fluids are infused into the patient prior to a surgical procedure, during the surgical procedure, or after the surgical procedure.

It is a further objective of the invention to provide a medical device which prevents infusion line disconnection at port sites during a medical procedure in which fluids are infused into the patient prior to a surgical procedure, during the surgical procedure, or after the surgical procedure.

It is yet another objective of the invention to provide a medical device which reduces the risk of infusion line connection unscrewing and becoming loose at port sites during a medical procedure in which fluids are infused into the patient prior to a surgical procedure, during the surgical procedure, or after the surgical procedure.

It is a still further objective of the invention to provide a medical device which minimizes the risk of infusion line connection unscrewing and becoming loose at port sites during a medical procedure in which fluids are infused into the patient prior to a surgical procedure, during the surgical procedure, or after the surgical procedure.

It is a further objective of the invention to provide a medical device which prevents infusion line connection unscrewing and becoming loose at port sites during a medical procedure in which fluids are infused into the patient prior to a surgical procedure, during the surgical procedure, or after the surgical procedure.

It is a still further objective of the invention to provide a medical device which minimizes the risk of infusion line connection leaking during a medical procedure in which fluids are infused into the patient prior to a surgical procedure, during the surgical procedure, or after the surgical procedure.

It is yet another objective of the invention to provide a medical device which reduces the risk of infusion line connection leaking during a medical procedure in which fluids are infused into the patient prior to a surgical procedure, during the surgical procedure, or after the surgical procedure.

It is a still further objective of the invention to provide a medical device which prevents infusion line leaking at port sites during a medical procedure in which fluids are infused into the patient prior to a surgical procedure, during the surgical procedure, or after the surgical procedure.

It is a further objective of the invention to provide a medical device that provides a mechanism for ensuring infusion line connections are properly connected.

It is yet another objective of the invention to provide a surgical device to prevent patients from awakening during a surgical procedure requiring anesthesia.

It is a still further objective of the invention to provide a surgical device which reduces the likelihood a patient awakens during a surgical procedure requiring anesthesia.

It is a further objective of the invention to provide a surgical device which minimizes the likelihood a patient awakens during a surgical procedure requiring anesthesia.

It is yet another objective of the invention to provide a surgical device to prevent patients from receiving inadequate perfusion during a surgical procedure requiring anesthesia.

It is a still further objective of the invention to provide a surgical device which reduces the likelihood a patient receives inadequate perfusion during a surgical procedure requiring anesthesia.

It is a further objective of the invention to provide a surgical device which minimizes the likelihood a patient receives inadequate perfusion during a surgical procedure requiring anesthesia.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1 is a front perspective view of an illustrative embodiment of a medical clip for use with an intravenous fluid infusion line;

FIG. 2 is an alternative front perspective view of the intravenous fluid infusion line clip illustrated in FIG. 1;

FIG. 3 is a back perspective view of the intravenous fluid infusion line clip illustrated in FIG. 1;

FIG. 4 is an alternative back perspective view of the intravenous fluid infusion line clip illustrated in FIG. 1;

FIG. 5 is a top view of the intravenous fluid infusion line clip illustrated in FIG. 1;

FIG. 6 is a bottom view of the intravenous fluid infusion line clip illustrated in FIG. 1;

FIG. 7 is a bottom perspective view of the intravenous fluid infusion line clip illustrated in FIG. 1;

FIG. 8 is a left side view of the intravenous fluid infusion line clip illustrated in FIG. 1;

FIG. 9 is a right side view of the intravenous fluid infusion line clip illustrated in FIG. 1;

FIG. 10 is a front view of the intravenous fluid infusion line clip illustrated in FIG. 1;

FIG. 11 is a back view of the intravenous fluid infusion line clip illustrated in FIG. 1;

FIG. 12 is a front view of the intravenous fluid infusion line clip, illustrating an embodiment of the inner surface of a fluid line engagement member;

FIG. 13 illustrates the intravenous fluid infusion line clip secured to an intravenous fluid infusion line;

FIG. 14 illustrates a connector line with a male connector port and a female connecting port at a y-site of the main fluid infusion line, shown in an unconnected position;

FIG. 15 illustrates a connector line with a male connector port and a female connecting port at a y-site of the main fluid infusion line, shown in a first connected position (spring partially compressed);

FIG. 16 illustrates a connector line with a male connector port and a female connecting port at a y-site of the main fluid infusion line, shown in a second connected position (spring fully compressed);

FIG. 17 illustrates the intravenous fluid infusion line clip attached to and securing two connected infusion lines; and

FIG. 18 is an alternative view of the intravenous fluid infusion line clip shown in FIG. 17.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

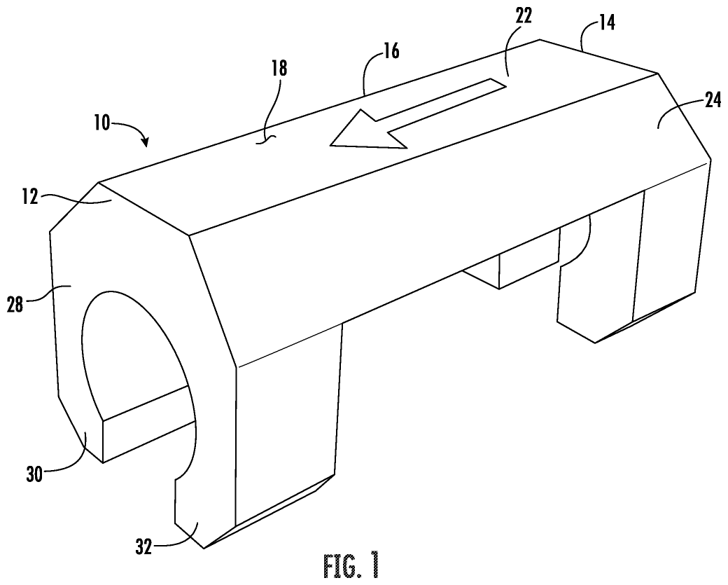

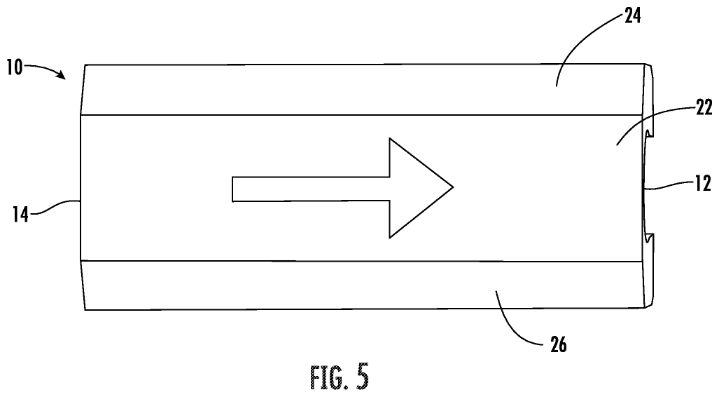

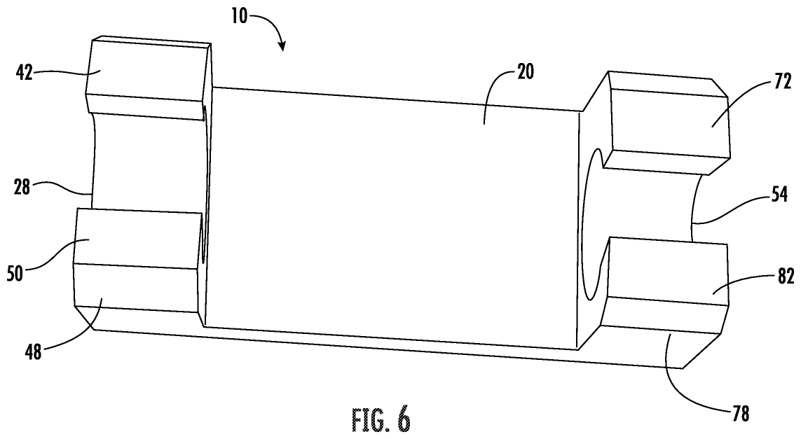

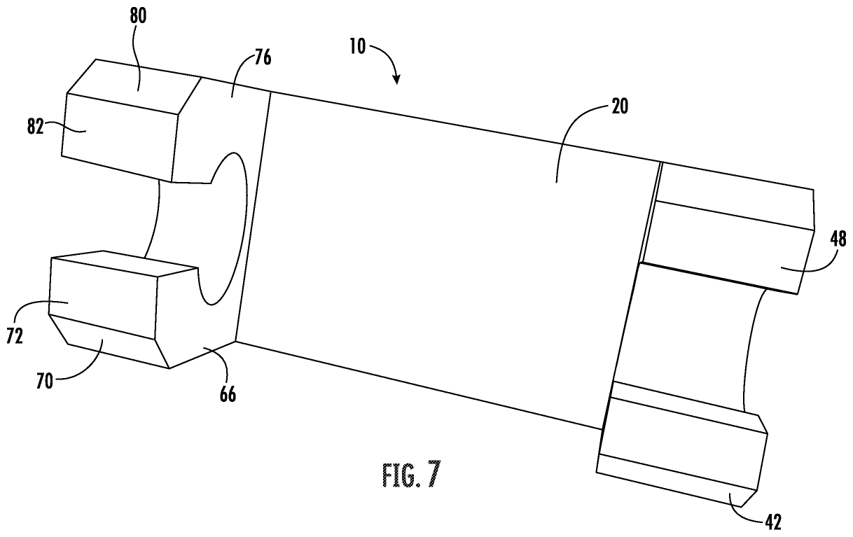

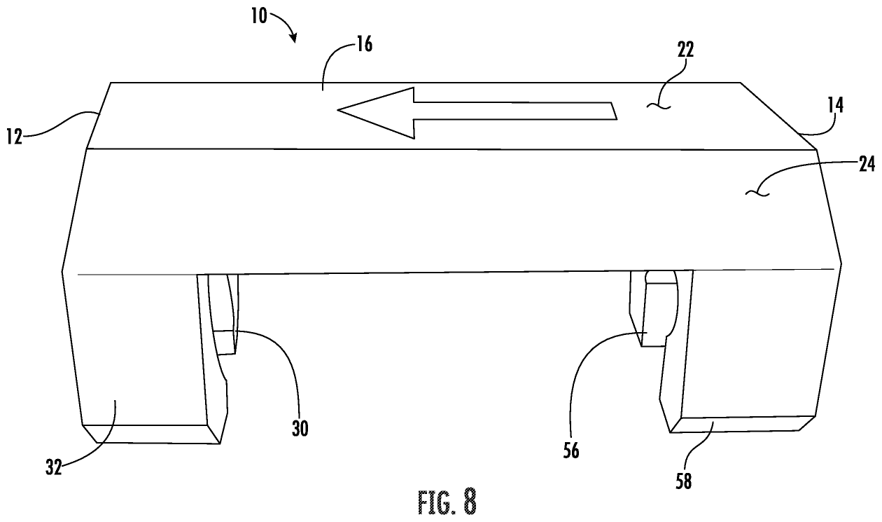

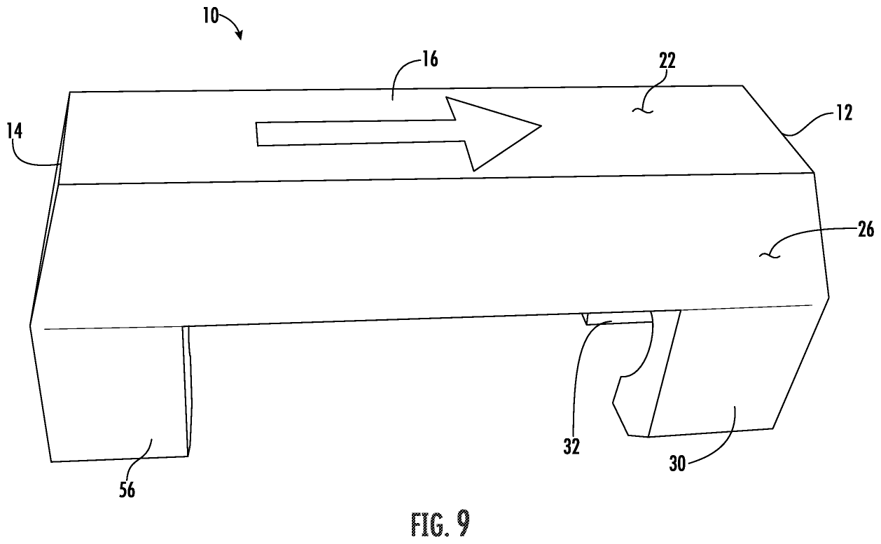

Referring to FIGS. 1-11, an illustrative example of a medical intravenous fluid infusion lines, referred to generally as an intravenous fluid line clip 10, is provided. The intravenous fluid line clip 10 comprises a first end 12, a second opposing end 14, and a main body 16 separating the first end 12 and the second opposing end 14 by a length. The length of separation is preferably sufficient to allow to allow portions of the intravenous fluid infusion lines, particularly areas of connections between two lines, such as male connector, male connector connected with corresponding female connector, to fight within and prevent separation of the connected components. The main body 16 comprises an outer surface 18 and an inner surface 20 (see FIGS. 6 and 7). The outer surface 18 may be configured to include a first generally planar surface or wall 22, a first slanted or angled surface or wall 24, and a second slanted or angled surface or wall 26. The first slanted surface or wall 24 diverges from or extends away from the first generally planar surface 22 at an angle. The second slanted surface or wall 26 diverges from or extends away from the first generally planar surface or wall 22 at an angle. Alternatively, the main body outer surface 18 may comprise a single, generally planar outer surface or wall 22.

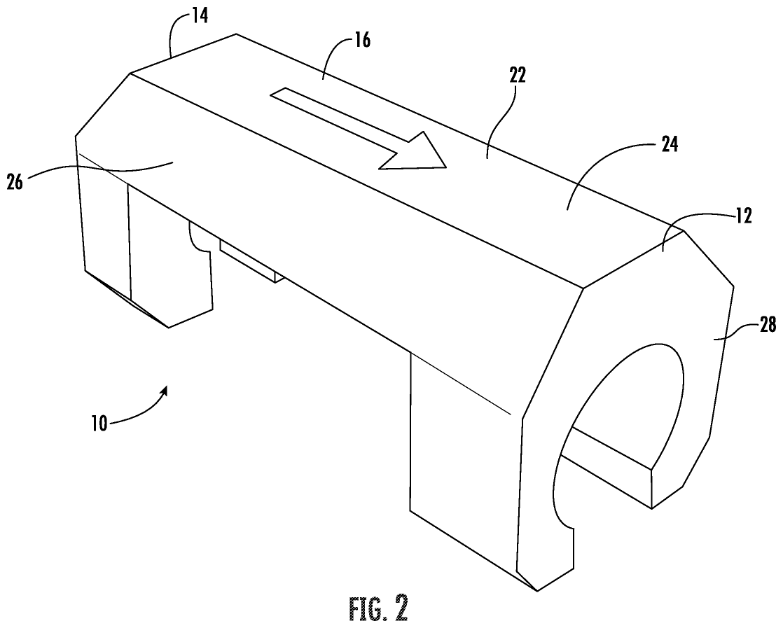

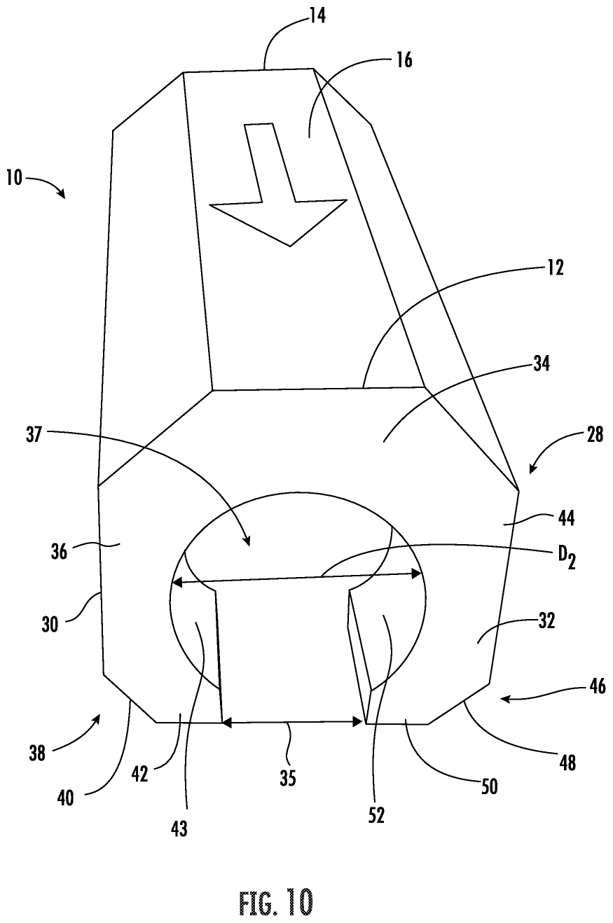

Integrally formed from, or attached to the first end 12, is a first fluid line engagement member 28 (see FIGS. 1 and 2). The first fluid line engagement member 28 is constructed and arranged to engage with and secure to a portion of an intravenous fluid line. The first fluid line engagement member 28 comprises a first arm 30, a second arm 32, and a bridging surface 34 connecting the first arm 30 and the second arm 32 (see FIG. 10). The bridging surface 34 may be formed by the end surfaces of the first generally planer surface 22, the first slanted surface 24, and the second slanted surface 26. The first arm 30 and the second arm 32 are separated by a space or distance 35. The first arm 30, the second arm 32, and the bridging surface 34 are arranged to provide an opening 37 at the first end 12 of sufficient size and shape to receive and engage with the intravenous fluid line. As illustrated, the opening 37 assumes a generally “C” shape.

The first arm 30 may be defined by an elongated, generally vertically orientated wall 36 terminating in a tip 38, see FIG. 10; the tip 38 having a first angled portion or surface 40. The first angled portion or surface 40 terminates in a generally planar, horizontally orientated portion or wall 42, thus forming, in combination with the first angled portion or surface 40, an inwardly directed, i.e. toward the center, portion. The positioning and shape of the vertically orientated wall 36 and the tip 38 provide an interior surface 43 having a curvature. The curvature of the interior surface 43 is preferably shaped to mirror the shape of a conjugate shaped tube of the intravenous fluid line it engages with.

The second arm 32 may be defined by an elongated, generally vertically orientated wall 44 terminating in a tip 46, see FIG. 10. The tip 46 has a first angled portion or surface 48. The first angled portion or surface 48 terminates in a generally planar, horizontally orientated portion or wall 50 thus forming, in combination with the first angled portion or surface 48, an inwardly directed, i.e. toward the center, portion. The positioning and shape of the vertically orientated wall 44 and the tip 46 provide an interior surface 52 having a curvature. The curvature of the interior surface 52 is preferably shaped to mirror the shape of a conjugate shaped tube of the intravenous fluid line it engages with.

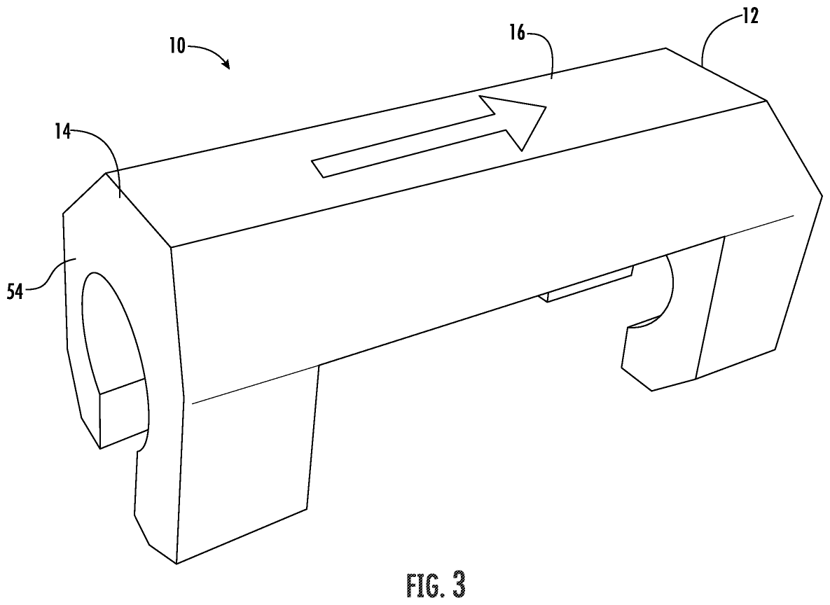

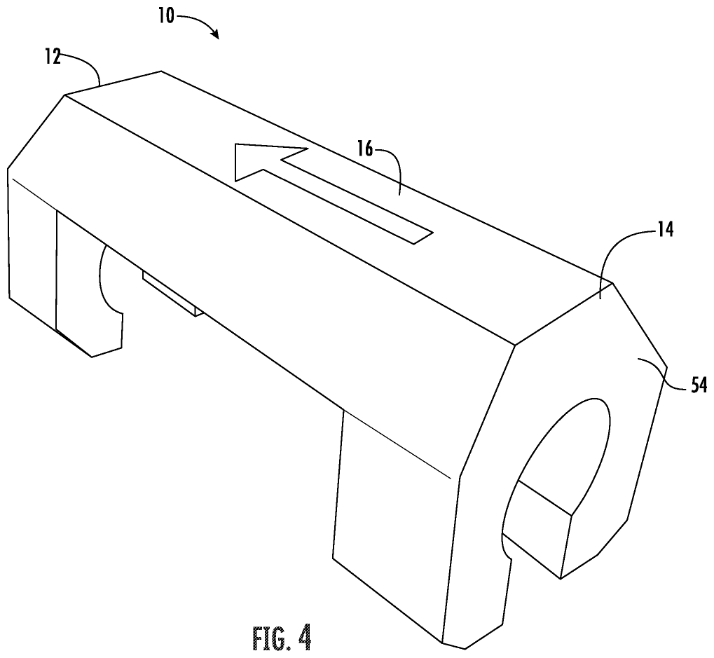

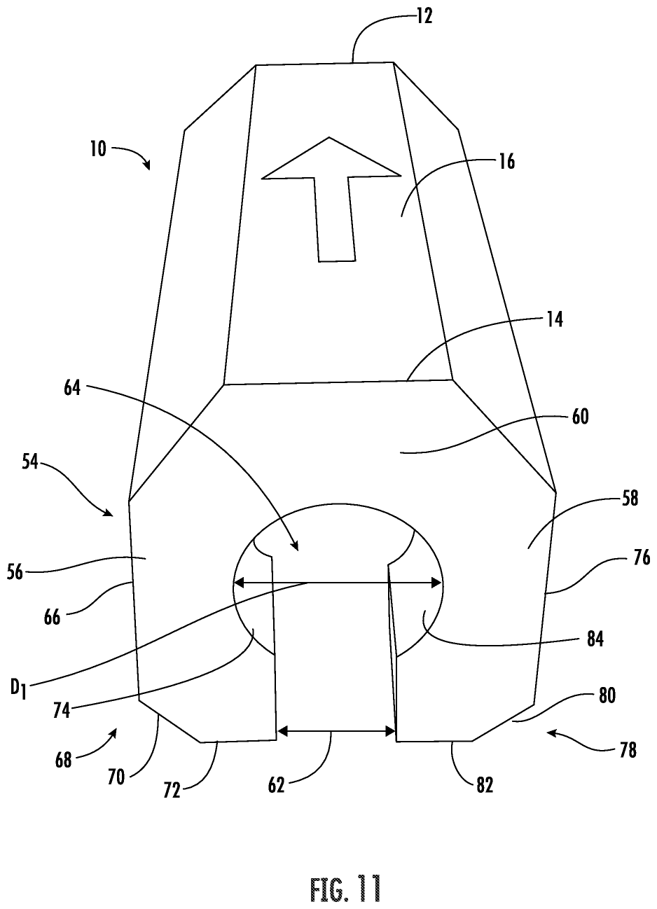

Integrally formed from, or attached to the second end 14, is a second fluid line engagement member 54 (see FIGS. 3 and 4). The second fluid line engagement member 54 is constructed and arranged to engage with and secure to a portion of the intravenous fluid line (different portion than where the first fluid line engagement member 28 secures to). The second fluid line engagement member 54 comprises a first arm 56, a second arm 58, and a bridging surface 60 connecting the first arm 56 and the second arm 58 (see FIG. 11). The bridging surface 60 may be formed by the end surfaces of the first generally planer surface or wall 22, the first slanted or angled surface or wall 24, and the second slanted or angled surface or wall 26. The first arm 56 and the second arm 58 are separated by a space 62. The first arm 56, the second arm 58, and the bridging surface 60 are arranged to provide an opening 64 at the second end 14 of sufficient size and shape to receive and engage with the intravenous fluid line. The opening 64 is shown assuming a generally “C” shape. In an illustrative example, opening 64 has a diameter D1 which is smaller than a diameter, D2, of opening 37. The different diameters allow the intravenous fluid line clip 10 to engage with and secure to sections of the intravenous fluid line that have different sizes. Alternatively, D1 could be the same diameter/length as that of D2.

The first arm 56 may be defined by an elongated, generally vertically orientated wall 66 terminating in a tip 68, see FIG. 11. The tip 68 has a first angled portion or surface 70. The first angled portion or surface 70 terminates in a generally planar, horizontally orientated portion or wall 72 thus forming, in combination with the first angled portion or surface 70, an inwardly directed, i.e. toward the center, portion. The positioning and shape of the vertically orientated wall 66 and the tip 68 provide an interior surface 74 having a curvature. The curvature of the interior surface 74 is preferably shaped to mirror the shape of a conjugate shaped tube of the intravenous fluid line it engages with.

The second arm 58 may be defined by an elongated, generally vertically orientated wall 76 terminating in a tip 78, see FIG. 11. The tip 78 has a first angled portion or surface 80. The first angled portion or surface 80 terminates in a generally planar, horizontally orientated portion or wall 82 thus forming, in combination with the first angled portion or surface 80, an inwardly directed, i.e. toward the center, portion. The positioning and shape of the vertically orientated wall 76 and the tip 80 provide an interior surface 84 having a curvature. The curvature of the interior surface 84 is preferably shaped to mirror the shape of a conjugate shaped tube of the intravenous fluid line it engages with.

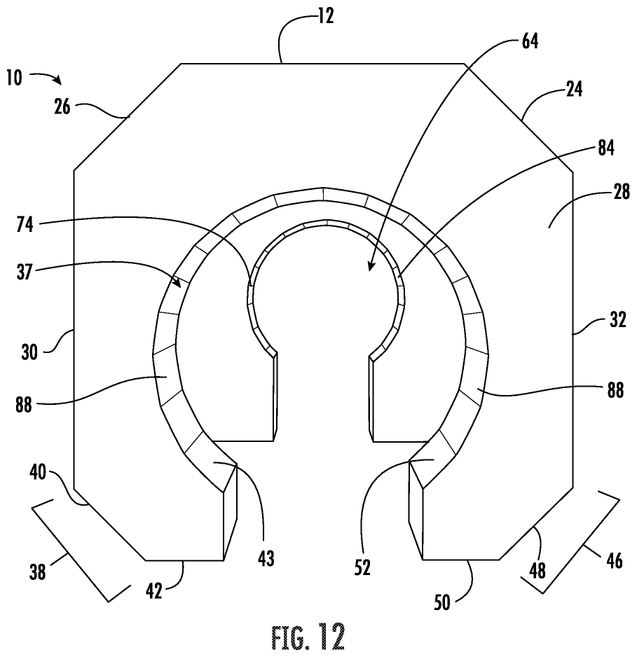

Referring to FIG. 12, an embodiment of the first fluid line engagement member 28 is shown having the inner surfaces 43 and 52 comprising a plurality of segmented surfaces 88. Each of the segmented surfaces 88 are orientated at an angle relative to an adjacent segment surface 88. The segmented surface 58 may be formed in which each segment is orientated at an angle relative to adjacent, individual sections. The second fluid line engagement member 54 may be configured in the same manner.

The intravenous fluid line clip 10 is designed for use with fluid infusion lines. The intravenous fluid line clip 10 may be used to attach to two primary ALARIS IV infusion lines or an ALARIS syringe pump IV line to a primary infusion set. In particular, the intravenous fluid line clip 10 is useful in cases requiring Total Intravenous Anesthesia (TIVA) and/or surgeries where access to intravenous (IV) connections is difficult. For example, during a carotid endarterectomy, the patient's arms are tucked and operating room (OR) staff is working up near the neck, and perfusion to the brain relies on the adequate connection of vasopressor medications. As part of the surgical lean against the patient's arms, which are tucked under sterile drapes, because they need to get as close to the patient as possible. Such action can result in loosening or disconnecting of IV connections. As the IV line may be under the sterile drapes, disconnection may not be found until it is too late. The intravenous fluid line clip 10 acts to decrease the risk of the connection unscrewing and becoming loose, as well as acting to avoid a leak or disconnection, even when the connection is unsecured by way of pressure on each end.

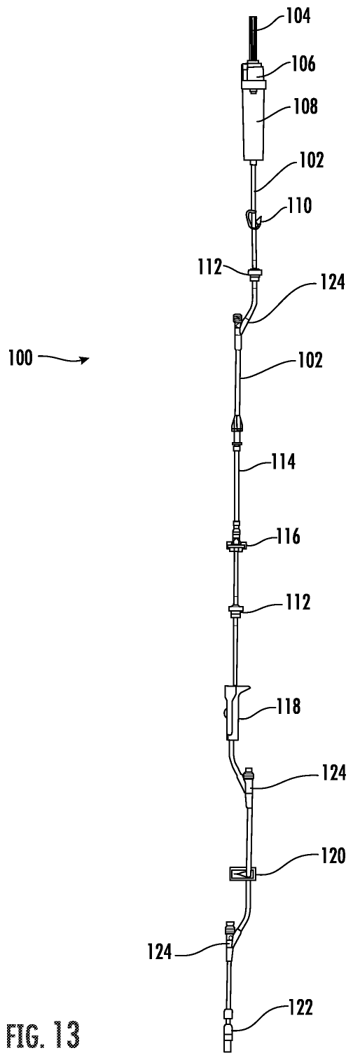

Referring to FIG. 13, an illustrative embodiment of an ALARIS Pump Infusion set (Becton Dickinson), referred to as infusion set 100, is shown. The infusion set 100 may comprise a main tubing line 102 sized and shaped for carrying a fluid, a bag spike 104, a vent cap 106, a drip chamber 108, a patch clamp 110, a back-check valve 112, a pump segment 114, a safety clamp 116, a roller clamp 118, a slide clamp 120, and a male luer 122. Along the length of the main tubing line 102, multiple y-sites 124 are provided. The y-sites 124 allow for connection of additional infusion lines to the main tubing line 102. The additional infusion lines carry different liquids, thus allowing a user to inject one or more mendicants anywhere along the main tubing line 102 through the y-sites 124.

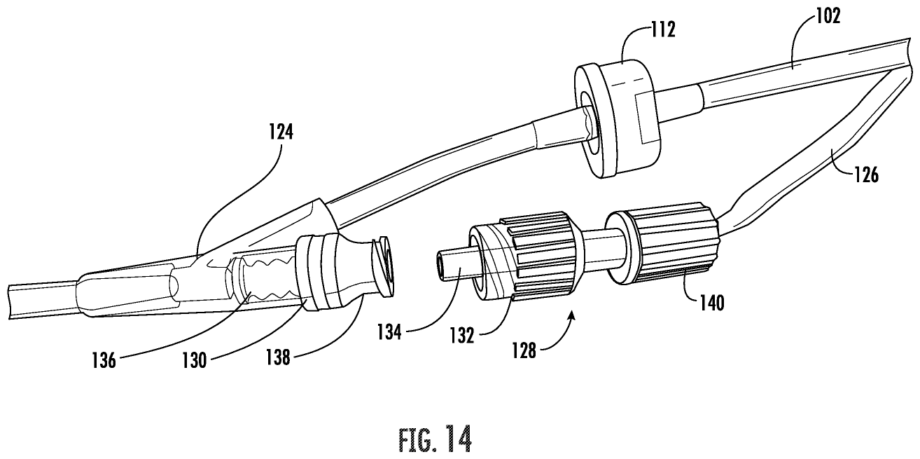

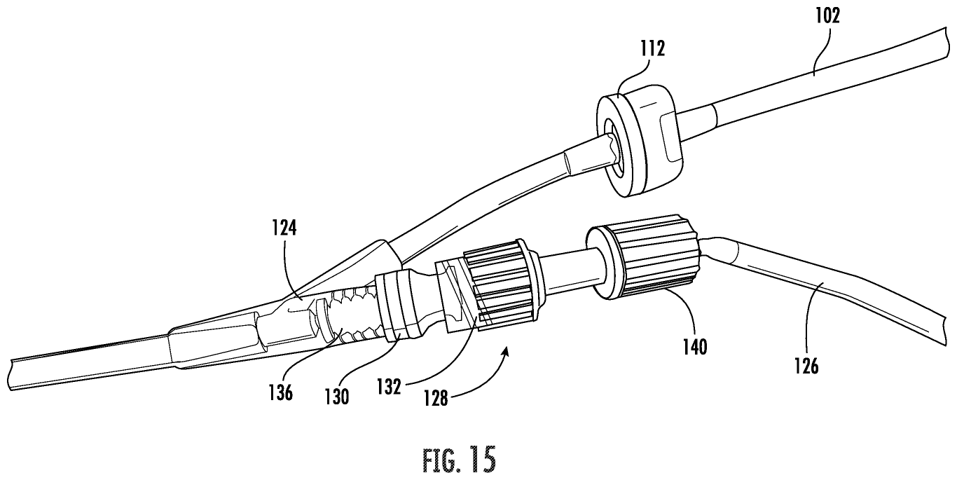

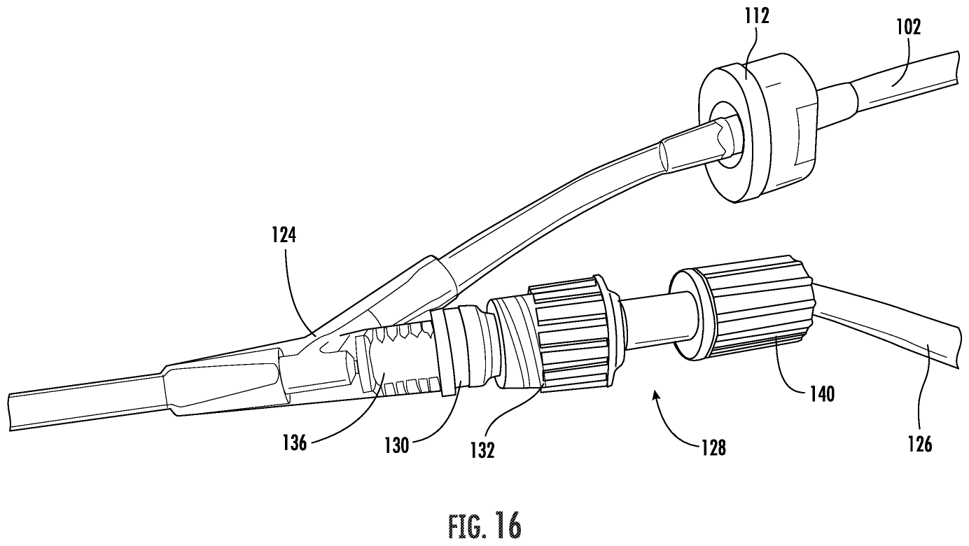

FIGS. 14-16 illustrate a connector line 126 with a male connector port 128. The connector line 126 connects to the main tubing line 102 at the y-site 124 via a spring-loaded female connector port 130. The male connector port 128 may include a threaded locking head 132 which is movable about an elongated insertion member 134. The elongated insertion member 134 is inserted into the spring-loaded female connector port 130, compressing spring 136, see FIGS. 15 and 16. As the spring 136 is compressed, the threaded locking head 132 is secured to the threaded head 138 of the spring-loaded female connector port 130. The male connector port 128 may include a stop member 140 (shown as a cylindrical body) to prevent the threaded locking head 132 from moving too far along the connector line 126. In use, the male connector port 128 secures to the spring-loaded female connector port 130 to prevent disconcertion of the connector line 126 and the main tubing line 102.

When properly secured, the male connector port 128-spring-loaded female connector port 130 connection is designed to prevent leaking. However, during the surgical procedure, patient awareness or inadequate perfusion may occur as a result of 1) provider error, or 2) disconnections at port sites. Use of the intravenous fluid line clip 10 prevents or decreases the risk of disconnection, thus secondarily reducing provider error. During a surgical procedure, attaching the intravenous fluid line clip 10 to the main tubing line 102 of the infusion set 100 acts as a fail-safe mechanism, ensuring the health care provider double checks the connection prior to placing the intravenous fluid line clip 10 thereon. The intravenous fluid line clip 10 then acts to prevent, minimize, or decrease the risk of the connection unscrewing and becoming loose, and acts to avoid a leak or disconnection even when the connection is unsecured by way of pressure on each end.

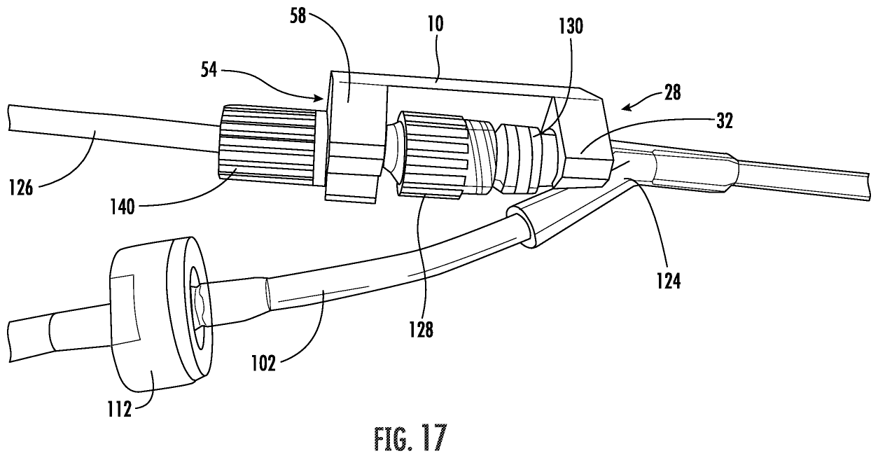

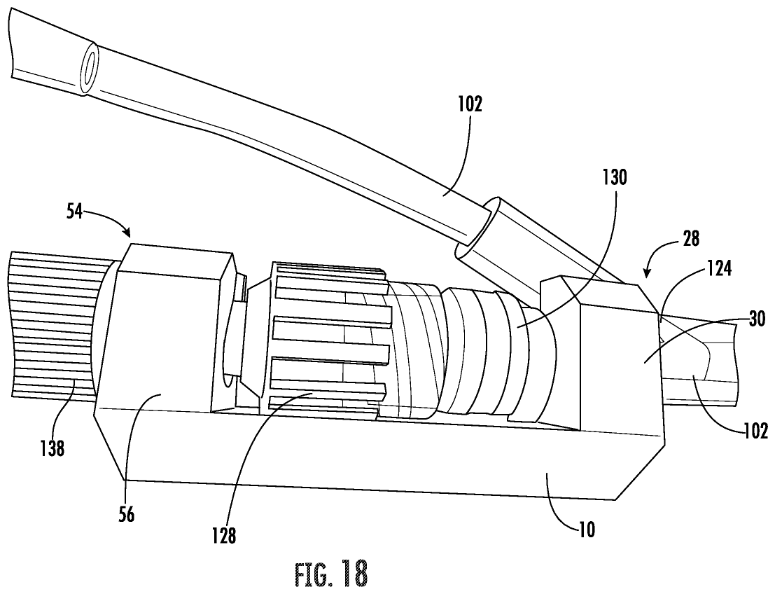

Referring to FIGS. 17 and 18, the intravenous fluid line clip 10 is shown secured to the connector line 126 at one end, and the at the y-site 124 of the main tubing line 102 at another end. To do so, the first fluid line engagement member 28 attaches to and secures to a portion of the main tubing line 102 (at or near the y-site 124), and the second fluid line engagement member 54 attaches and secures along one section of the connector line 126. Because the first fluid line engagement member 28 is sized larger than the second fluid line engagement member 54, proper placement is directional (first fluid line engagement member 28 securing to the wider diameter portions of the tuning line; the second fluid line engagement member 54 securing to the smaller diameter portions of the tuning line). The first fluid line engagement member 28 secures to a portion of the main tubing line 102 via opening 37, with the tubing 102 resting between the first fluid line engagement member first arm 30 and the first fluid line engagement member second arm 32. Both arms 30 and 32 rest just under the spring-loaded female connector port 130.

The second fluid line engagement member 54 secures to a portion of the connector line 126 via opening 64. A portion of the connector line 126 rests between the second fluid line engagement member first arm 56 and the second fluid line engagement member second arm 58. Both arms 56 and 58 secure around the tubing between the male connector port threaded locking head 132 and the male connector port stop member 140. In this position, disengagement of the connector line 126 from the main tubing line 102 is minimized or prevented, even if there is partial unthreading due to connection error or patient movement of the patient's body during the procedure.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures, and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A surgical clip for preventing separation of connected fluid lines, comprising:
a first end having a first fluid line engagement member;
a second opposing end having a second fluid line engagement member; and
a body separating said first end and said second end

2. The surgical clip for preventing separation of connected fluid lines according to claim 1, wherein,
said first fluid line engagement member comprises:
a first arm;
a second arm;
wherein, said first arm is separated from said second arm by a space or distance defining a first opening, said first opening of sufficient size and shape to receive and engage with a first portion of an intravenous fluid line; and
said second fluid line engagement member comprises:
a first arm;
a second arm;
wherein, said first arm is separated from said second arm by a space or distance defining a second opening, said second opening of sufficient size and shape to receive and engage with a second portion of said intravenous fluid line.

3. The surgical clip for preventing separation of connected fluid lines according to claim 2, wherein said first opening is defined by an interior surface, said interior surface having a shape that mirrors the shape of said intravenous fluid line it engages with.

4. The surgical clip for preventing separation of connected fluid lines according to claim 2, wherein said second opening is defined by an interior surface, said interior surface having a shape that mirrors the shape of said intravenous fluid line it engages with.

5. The surgical clip for preventing separation of connected fluid lines according to claim 2, wherein said first opening has a different diameter than a diameter of said second opening.

6. The surgical clip for preventing separation of connected fluid lines according to claim 2, wherein said first fluid line engagement member first arm comprises a terminal end having an inwardly directed portion, and said first fluid line engagement member second arm comprises a terminal end having an inwardly directed portion.

7. The surgical clip for preventing separation of connected fluid lines according to claim 2, wherein said second fluid line engagement member first arm comprises a terminal end having an inwardly directed portion, and said second fluid line engagement member second arm comprises a terminal end having an inwardly directed portion.

8. The surgical clip for preventing separation of connected fluid lines according to claim 1, wherein said body comprises an upper surface.

9. The surgical clip for preventing separation of connected fluid lines according to claim 8, wherein said body upper surface comprises at least one angled surface.

10. The surgical clip for preventing separation of connected fluid lines according to claim 1, wherein said body upper surface is defined by two angled surfaces separated by a generally planer surface.

11. A system for preventing separation of connected fluid lines comprising:
a surgical clip for preventing separation of connected fluid lines, comprising: a first end having a first fluid line engagement member; a second end having a second fluid line engagement member; and a body separating said first end and said second end; and
a fluid line configured to receive a fluid.

12. The system for preventing separation of connected fluid lines according to claim 11, wherein,
said first fluid line engagement member comprises:
a first arm;
a second arm;
wherein, said first arm is separated from said second arm by a space or distance defining a first opening, said first opening of sufficient size and shape to receive and engage with a first portion of an intravenous fluid line; and
said second fluid line engagement member comprises:
a first arm;
a second arm;
wherein, said first arm is separated from said second arm by a space or distance defining a second opening, said second opening of sufficient size and shape to receive and engage with a second portion of said intravenous fluid line.

13. The system for preventing separation of connected fluid lines according to claim 12, wherein said first opening has a different diameter than a diameter of said second opening.

14. The system for preventing separation of connected fluid lines according to claim 12, wherein
said first fluid line engagement member first arm comprises a terminal end having an inwardly directed portion;
said first fluid line engagement member second arm comprises a terminal end having an inwardly directed portion;
said second fluid line engagement member first arm comprises a terminal end having an inwardly directed portion; and
said second fluid line engagement member second arm comprises a terminal end having an inwardly directed portion.

15. The system for preventing separation of connected fluid lines according to claim 11, wherein said fluid line comprises one or more connecting sites having a connector port.
